(19) United States
(12) Patent Application Publication
(10) Pub. No.: US 2023/0060345 A1
(43) Pub. Date: Mar. 2, 2023

(54) LASER-BASED SELECTIVE BTEX SENSING WITH DEEP NEURAL NETWORK

(71) Applicant: KING ABDULLAH UNIVERSITY OF SCIENCE AND TECHNOLOGY, Thuwal (SA)

(72) Inventors: Aamir FAROOQ, Thuwal (SA); Mhanna MHANNA, Thuwal (SA); Mohamed SY, Thuwal (SA)

(21) Appl. No.: 17/901,068

(22) Filed: Sep. 1, 2022

Related U.S. Application Data
(60) Provisional application No. 63/239,558, filed on Sep. 1, 2021.

(51) Int. Cl. G01N 21/3504; G01N 33/00

(52) U.S. Cl. CPC G01N 21/3504; G01N 33/0047; G01N 2201/06113

(57) ABSTRACT

A laser-based detection and analysis system for detecting plural members of volatile organic compounds includes a measuring unit configured to simultaneously measure a spectrum of the plural members of the volatile organic compounds located in a measuring chamber, with a laser beam having a wavelength of about 3.3 μm, and a data processing unit including a deep neural network, DNN, configured to process the spectrum measured by the measuring unit and to output an individual concentration of each of the plural members of the volatile organic compounds. The DNN is configured to update a weight Wₖ for each member of the plural members by using hidden layers having plural nodes, each node having an activation function and an optimizer.

### CROSS-REFERENCE TO RELATED APPLICATIONS

This application claims priority to U.S. Provisional Patent Application No. 63/239,558, filed on Sep. 1, 2021, entitled “LASER-BASED SELECTIVE BTEX SENSING USING DEEP NEURAL NETWORKS,” the disclosure of which is incorporated herein by reference in its entirety.

### BACKGROUND

### Technical Field

Embodiments of the subject matter disclosed herein generally relate to a system for simultaneously detecting and distinguishing between benzene, toluene, ethylbenzene and xylene isomers (BTEX) in a given mixture, and more specifically, to a laser-based system that uses a laser beam for measuring the spectrum of the BTEX members and a deep neural network (DNN) for distinguishing each member of the BTEX family.

### Discussion of the Background

Human activities, such as those related to transportation and petrochemicals, can have negative impacts on the air quality. The petrochemical industries are the major emitters of volatile organic compounds (VOCs). In particular, benzene, toluene, ethylbenzene and xylene isomers are emitted from engine exhausts, gasoline service stations, refineries, paint and rubber industries.

The BTEX members have severe negative health effects on humans. For instance, short exposure (5-10 min) to large amounts of benzene (10,000-20,000 ppm) can lead to death, while 70-3000 ppm exposure can cause unconsciousness or dizziness. Daily exposure to 10 ppm of benzene for several hours can cause neurological dysfunction over long term. Inhaling 600-5000 ppm of toluene can damage the brain, liver and kidneys of a healthy individual. Irritation of eyes and respiratory tract have been reported after exposure to 1000-5000 ppm of ethylbenzene for a few seconds. It has been found that the three isomers of xylene have similar health effects, and exposure to 700-10,000 ppm xylene can cause a lack of muscle coordination and distortion to the nervous system.

Particularly the benzene (C₆H₆), also known as benzol, is a colorless liquid with a sweet odor. Benzene evaporates into air very quickly and dissolves slightly in water. Benzene is highly flammable. Most people can begin to smell benzene in air at approximately 60 parts of benzene per million parts of air (ppm) and recognize it as benzene at 100 ppm. Most people can begin to taste benzene in water at 0.5- 4.5 ppm. One part per million is approximately equal to one drop in 40 gallons. Benzene is found in air, water, and soil.

To prevent or be aware of benzene and other BTEX members contamination, various sensors are currently used at the chemical processing facilities for determining any escape of these chemicals into the environment. However, due to the similar spectrum of the BTEX members, it is difficult with the current systems to distinguish between the various BTEX members when they are simultaneously present in air. For this reason, there is a large demand for new and improved BTEX sensors as the existing commercial sensors lack in many respects. It is desired to have reliable, accurate, sensitive, and real-time diagnostic methods and sensors for BTEX members detection.

Conventional techniques like gas chromatography, mass spectrometry and Fourier transform infrared spectroscopy involve expensive, bulky and complex instrumentation, and are not quite suitable for field analysis. Chemical and biosensors have been applied for the measurement of BTEX members. However, a sensing material with high sensitivity and selectivity is still challenging [1]. Therefore, there is still an acute need to develop accurate and portable sensors for BTEX members.

Laser absorption spectroscopy is a species-specific technique which enables highly sensitive and selective detection of target molecules. However, the similar absorption spectra of BTEX members in the IR wavelength region has been a barrier to developing laser-based absorption sensors for these aromatic molecules. Thus, there is a need to overcome this barrier and produce a simple, portable, laser-based mid-IR spectroscopic sensor for selective measurements and identification of BTEX members.

### SUMMARY

According to an embodiment, there is a laser-based detection and analysis system for detecting plural members of volatile organic compounds. The system includes a measuring unit configured to simultaneously measure a spectrum of the plural members of the volatile organic compounds located in a measuring chamber, with a laser beam having a wavelength of about 3.3 μm, and a data processing unit including a deep neural network, DNN, configured to process the spectrum measured by the measuring unit and to output an individual concentration of each of the plural members of the volatile organic compounds. The DNN is configured to update a weight Wₖ for each member of the plural members by using hidden layers having plural nodes, each node having an activation function and an optimizer.

According to another embodiment, there is a laser-based detection and analysis system for simultaneously detecting benzene, toluene, ethylbenzene, and xylenes. The system includes a laser device configured to emit a laser beam that includes a wavelength of 3.3 μm, a measuring chamber configured to receive, at an internal cavity, ambient air and the laser beam, wherein the measuring chamber is configured to bounce the laser beam inside the internal cavity multiple times before exiting the measuring chamber, a photosensor configured to receive an output laser beam from the measuring chamber, and a data processing unit including a deep neural network, DNN, which is configured to receive a measurement from the photosensor and to simultaneously detect an amount of the benzene, toluene, ethylbenzene, and xylenes in the ambient air.

According to yet another embodiment, there is a method for simultaneously detecting plural members of volatile organic compounds. The method includes a step of simultaneously measuring, within a measuring unit, a spectrum of the plural members of the volatile organic compounds located in a measuring chamber, with a laser beam having a wavelength about 3.3 μm, a step of providing the measured spectrum to a data processing unit that includes a deep neural network, DNN, and a step of calculating at the DNN individual concentrations of each of the plural members of the volatile organic compounds. The DNN is configured to update a weight Wₖ for each member of the plural members by using hidden layers having plural nodes, each node having an activation function and an optimizer.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic illustration of a laser-based detection and analyzing system for detecting plural members of volatile organic compounds;

FIG. 2 illustrates in more detail a measuring unit of the laser-based detection and analyzing system;

FIG. 3 illustrates the infrared spectra of the plural members of the volatile organic compounds for the C—H stretching band under normal atmospheric conditions;

FIG. 4 presents a table illustrating 10-fold cross-validation results on an assembled spectral database;

FIG. 5 schematically illustrates a structure of a deep neural network used by the laser-based detection and analyzing system and associated flow chart for using the network;

FIG. 6 is a flow chart of a method for using the laser-based detection and analyzing system with the deep neural network for simultaneously determining the presence and concentrations of the plural members of the volatile organic compounds;

FIG. 7 presents a table illustrating the ratios of the volatile organic compounds used to train the deep neural network;

FIGS. 8A to 8D illustrate the predicted mole fractions with manometric values for 12 mixtures using multi-dimensional linear regression;

FIGS. 9A to 9D illustrate the predicted mole fractions with manometric values for 12 mixtures using support vector machines;

FIGS. 10A to 10D illustrate the predicted mole fractions with manometric values for 12 mixtures using extreme gradient boosting;

FIGS. 11A to 11D illustrate the predicted mole fractions with manometric values for 12 mixtures using deep neural networks;

FIG. 12 illustrates real-time measurements of 4400 ppm BTEX/N₂ mixture at ambient conditions; and

FIG. 13 illustrates a computing system in which the deep neural network may be implemented.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. For simplicity, the following embodiments are discussed with regard to a portable, laser-based, mid-IR spectroscopic sensor for selective measurements of BTEX species and a DNN based analyzer that is capable to distinguish between the various members of the BTEX members. However, the embodiments discussed herein are not limited to such a laser-based sensor, but they may be applied to other sensors.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

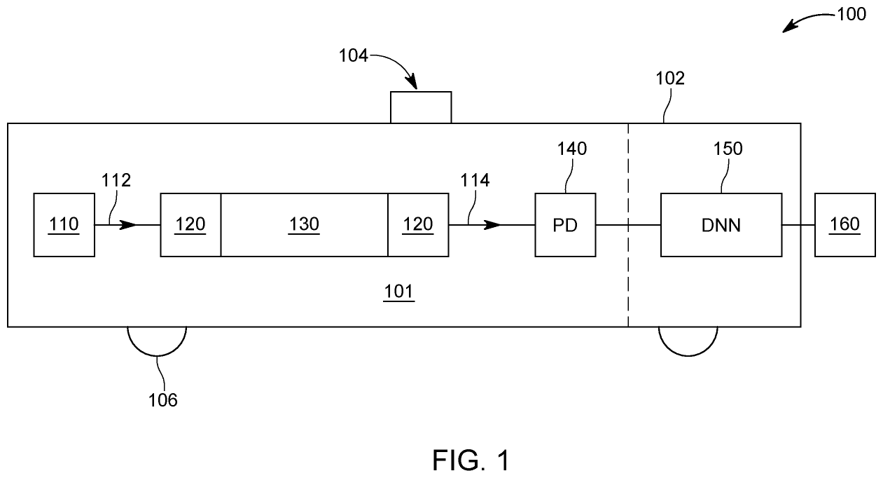

According to an embodiment, a portable, laser-based, selective BTEX sensing and analyzing system 100 is shown in FIG. 1 and has a measuring unit 101 and a data processing unit 150. The measuring unit 101 includes a laser device 110, optical elements 120, a measurement chamber 130, and a photosensor 140. The laser device 110 may emit a light beam 112, which is directed by the optical elements 120 to the measurement chamber 130. The light beam 112 is reflected inside a cavity (not shown) of the measurement chamber 130, multiple times, and an output light 114, which escapes from the measurement chamber 130, is detected by the photosensor 140. The light intensity measured by the photosensor 140 is transmitted to the data processing unit 150, where a DNN analysis is performed. Based on this analysis, the simultaneous presence of two or more of the BTEX members is determined and is then displayed on a monitor 160.

All these components of the system 100 (except the monitor 160) may be packaged into a common housing 102. The housing is small in dimensions so that a person can take the entire system 100 and move it to a desired location, i.e., the system 100 is portable. Note that the term “portable” is defined in this application to describe an object that can be carried by one or two persons, and not a large object that needs to be carried by a vehicle. The housing 102 may have a handle 104 so that the person can physically carry the system from one location to another. Alternately, the housing 102 may have one or more wheels 106 so that the entire system can be pushed by that person, on its wheels, to the desired location. In one application, the system 100 has both the handle 104 and the wheels 106.

As discussed in [2], which is assigned to the assignee of the present application, although benzene has absorption bands in the ultraviolet (UV) wavelength region, the broad features of most hydrocarbons in this region do not permit interference-free selective measurements. In other words, the benzene and many other hydrocarbons have similar signatures in the UV region and thus, the presence of one hydrocarbon cannot be distinguished from the presence of another hydrocarbon with the traditional sensors. For this reason, the laser device 110 is specifically selected/tuned to generate an infrared light beam as the infrared (IR) absorption spectrum of these hydrocarbons provide better opportunities for the highly selective detection of benzene and other pollutants.

Based on the IR spectrum of benzene, the best wavelength for detecting the benzene presence is near 674 cm⁻¹ (i.e., a wavelength of 14.837 μm). However, the inventors have observed in [2] that this wavelength region is currently not accessible by commercially available semiconductor lasers. The inventors have selected, for the embodiment illustrated in FIG. 1, to excite the laser device 110 to take advantage of the v20 vibrational band of benzene, which is near 3.3 μm, i.e., the laser device 110 is tuned to emit the laser beam 112 having a wavelength about 3.3 μm. In one application, a range of 2.5 to 4.0 μm is used for the wavelength of the laser device 110 for the analysis. In another application, a range of 3.0 to 4.5 μm is selected. Other ranges can be selected for the wavelength of the laser beam, as long as the range includes the 3.3 μm wavelength. The term “about” 3.3 μm is used herein to cover any of the ranges noted above, by specifically the 3.2889 to 3.2903 range.

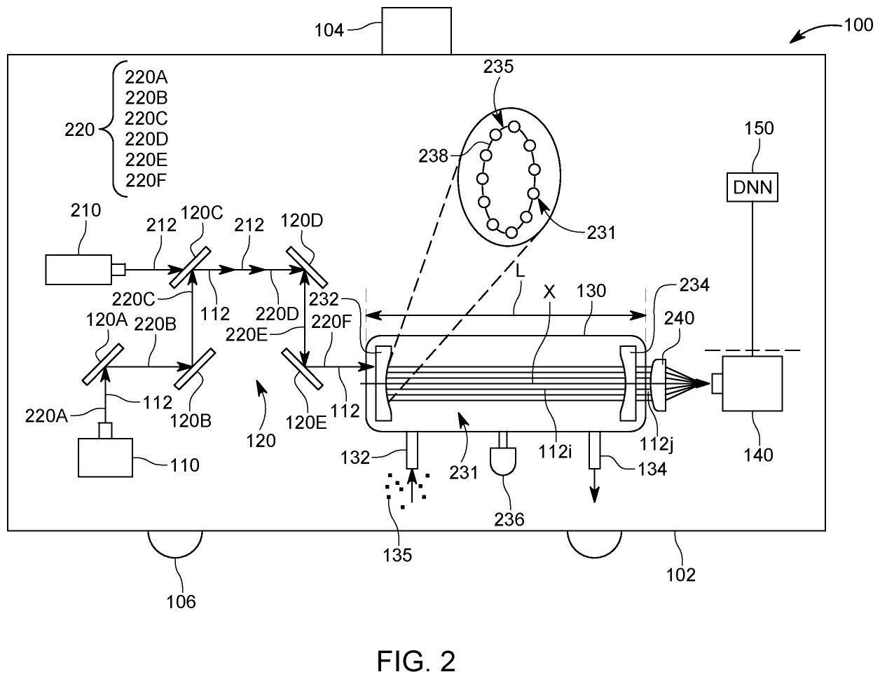

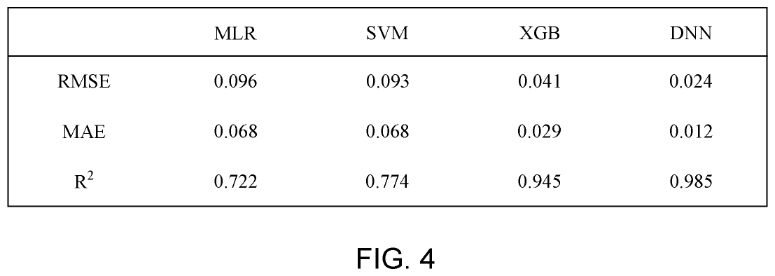

One possible implementation of the laser-based detection and analysis system 100 is now discussed with regard to FIG. 2, which partially corresponds to FIG. 4 from [2]. The system 100 includes the laser device 110 and the measurement chamber 130 shown in the embodiment of FIG. 1. The laser device 110 may be an interband cascade laser in this embodiment. An interband cascade laser is a device that produces coherent radiation over a large part of the mid-infrared region of the electromagnetic spectrum. The term “mid-infrared region” is understood herein to refer to a 3-8 μm wavelength band. For example, a DFB interband cascade laser (DFB-ICL, Nanoplus, Germany) emits near 3.3 μm with an output power of about 1-2 mW. However, any other laser or other light source that can emit a light beam with this wavelength may be used. In one application, the laser wavelength of the beam 112 was tuned over 3039.25-3040.5 cm⁻¹ by a linear ramp of injection current (1 kHz scan rate), and a 7.62 cm germanium etalon was utilized to convert the scan time to wavenumber.

The embodiment shown in FIG. 2 directs the emitted laser beam 112, with plural mirrors 120A to 120E, to the measurement chamber 130. The plural mirrors 120A to 120E are part of the optical components 120. This embodiment shows the use of 5 such mirrors. However, fewer or more mirrors may be used. These mirrors may be used to integrate an additional laser beam 212 from an additional laser device 210, called herein an alignment laser device. However, this additional laser device is optional. While the mirrors 120A, 120B, 120D and 120E are fixed mirrors, mirror 120C is a flip mirror as there might be the need, before using the laser beam 110 to detect the BTEX components, that various components of the laser-based detection and analysis system 100 need to be optically aligned. Note that the laser-based detection and analysis system 100 also includes mirrors 232 and 234 (which make the laser beam 112 to reflect multiple times inside the measurement chamber 130), located inside a cavity 231 formed inside the measurement chamber 130, a convergent lens 240, and the photosensor 140. As the main laser device 110's beam 112 is not visible (being in the infrared range), the alignment laser device 210 is selected to emit a visible laser beam 212 that can be used for alignment purposes. This is achieved with the flip mirror 120C. If no alignment is necessary, the additional laser device 210 and one or more of the mirrors 120A, 120B, 120D and 120E can be omitted.

After the laser beam 112 leaves the last mirror 120E, it enters inside the measurement chamber 130. The measurement chamber 130 includes two mirrors 232 and 234, located in the cavity 231, which are designed to reflect multiple times the incoming laser beam 112. In one application, two ZnSe mirrors of 99.97% nominal reflectivity (LohnStar Optics) are used to form the cavity 231 and the cavity has a length L (e.g., 30 cm). The length of the cavity can be modified. The laser beam 112 is aligned in an off-axis mode (i.e., not entering along a symmetry longitudinal axis X of the measurement cavity), which suppresses the spurious coupling noise compared to the on-axis cavity. A reflection pattern 235 of the incoming laser beam 112 inside the cavity 231 is illustrated in FIG. 2. FIG. 2 shows that the various points of reflections of the laser beam may be disposed on an elliptical path 238. Note that the figure shows the multiple reflections 112ᵢ of the incoming laser beam 112, between the two mirrors 232 and 234.

At each reflection on the mirror 234, part of the light 112ᵢ passes the mirror and exits from the measurement chamber 130 as output laser beams 112ⱼ. These output laser beams 112ⱼ are then collected via a focusing lens 240 (e.g., a convergent lens) on the photosensor 140. In one application, the photosensor 140 is a photodetector, for example, a 1.5 MHz AC-coupled, TE-cooled photodetector (Vigo Systems). The data collected at the photosensor 140 is then transmitted to the data processing unit 150 for processing.

FIG. 2 also shows that the measurement chamber 130 has an inlet port 132 and an outlet port 134 that fluidly communicate with the cavity 231. A pressure sensor 236 may be connected to the cavity 231 of the chamber 130 for measuring the pressure inside. The inlet and outlet ports are used to circulate ambient air 135 through the measurement chamber 130 for refreshing the air inside the cavity for achieving accurate measurements. In this way, a measurement of the BTEX members inside the measurement chamber 130 reflects the presence and amount of the various members in the ambient air.

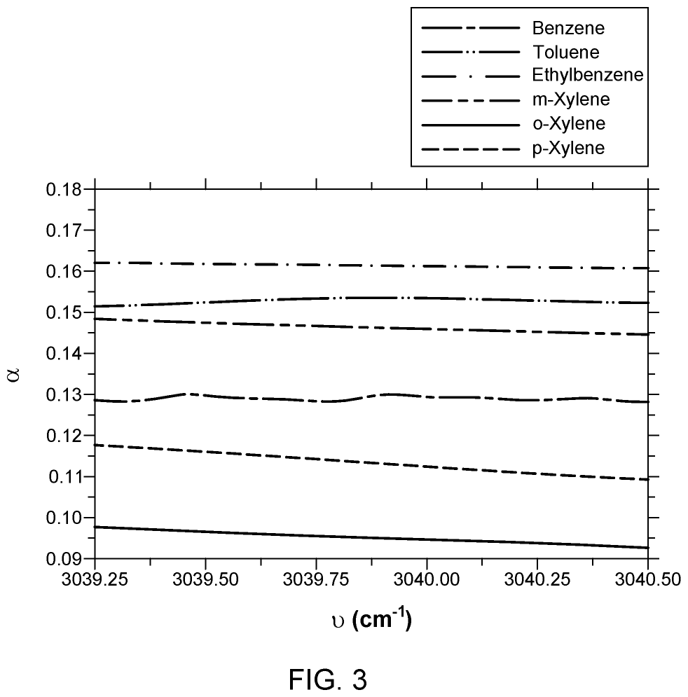

For the selected wavelength of 3.3 μm (3040 cm⁻¹), which corresponds to the C—H stretching bands in the BTEX members, a simulated absorbance spectra for some these members is presented in FIG. 3. It is noted that the absorption spectra is similarly shaped for all these elements, which make it difficult for the traditional algorithms to distinguish between individual members of the BTEX family. Multi-dimensional linear regression (MLR) was previously used by [2] to distinguish the spectra of benzene, ethylene and methane. However, the predictive ability of MLR is dramatically compromised in the case of BTEX members. Hence, various machine learning algorithms, such as support vector machines (SVMs), extreme gradient boosting (XGB) and deep neural networks (DNNs) were explored by the inventors. The general principles of these algorithms are well explained in literature [3-5] and thus, their description is omitted herein. To test these algorithms, the inventors assembled a database comprising 5400 simulated spectra and 132 measured spectra. The simulated spectra were generated using the Pacific Northwest National Laboratory (PNNL) spectral database, by randomly varying the concentrations of each BTEX species Xₖ (k describes the numbers of members or species of the BTEX family), in the range of 1-1000 ppm, while the total BTEX concentration, χₜₒₜₐₗ was fixed to 1000 ppm. It is noted that the total concentration does not need to be varied as the concentration ratios,

$$\frac{{χ}_{k}}{{χ}_{t o t a l}} ,$$

are normalized. For the measured spectra, plural mixtures were prepared in the lab with various concentration ratios where the total BTEX concentration varied within a range of 100-5000 ppm. Application of the chosen algorithms on the assembled database resulted in the 10-fold cross-validation results illustrated in the table of FIG. 4. The data were randomly split into 80/20 train/test sets. The best performance was achieved with a DNN containing three hidden layers with 64, 32 and 16 nodes, respectively.

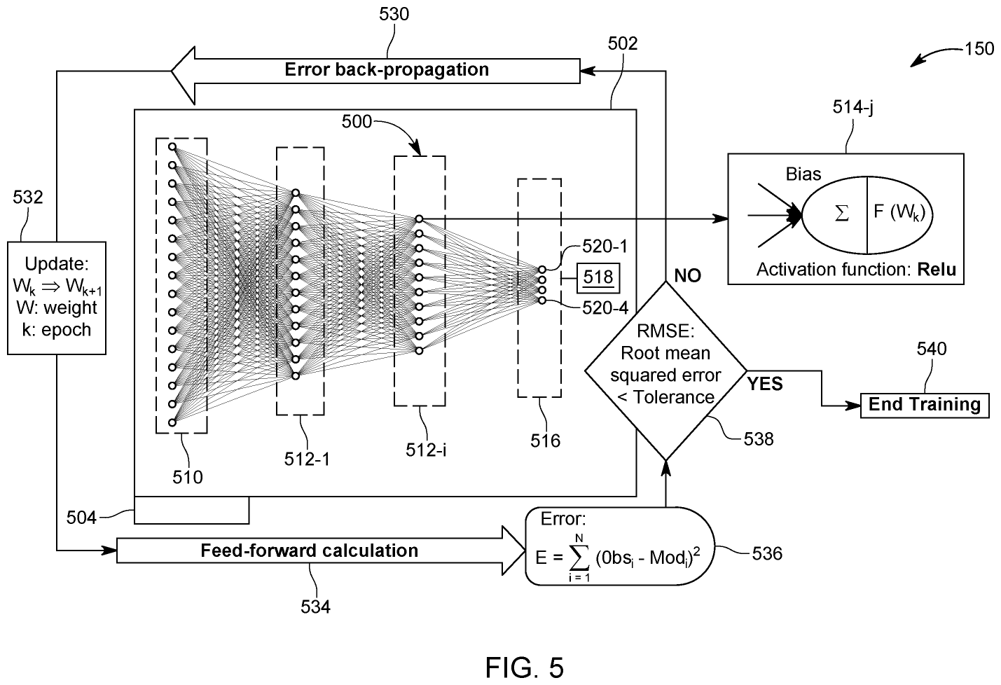

The specific structure/arrangement of the data processing unit 150 is illustrated in FIG. 5. The data processing unit 150 includes a processor 502 and a memory 504 that store a DNN 500. The DNN 500 includes an input layer 510 that is configured in this embodiment to have 5532 inputs, 5400 for the simulated BTEX absorbance spectra and 132 for the measured absorbance spectra. Note that any spectrum of the input spectra may contain data about one, two, three or all members of the BTEX family. In other words, it is possible that the system 100 simultaneously collects the spectrum of all members of the BTEX family if all these members are present in the input air 135. The input layer is connected to a first hidden layer 512-1. The DNN 500 may include a number N of hidden layers 512-i, where i takes values between 2 and N, and N is a positive integer equal to or larger than 3. In the embodiment discussed herein, N is 3. Each hidden layer 512-i includes plural neurons 514-j, where j is a positive integer, for example, 16, 32, or 64. In one embodiment, the first hidden layer 512-1 has 64 nodes, the second hidden layer has 32 nodes, and the third hidden layer has 16 nodes. Other values are possible. Each node in a given hidden layer is connected to all nodes from a previous hidden layer and all nodes of a next hidden layer. In one application, the neuron 514-j has an activation function ReLU (Rectified Linear Unit), as illustrated in FIG. 5. Note that the rectifier or ReLU activation function is an activation function defined as the positive part of its argument: f (x)=x⁺=max (0, x) where x is the input to a neuron. The last hidden layer is connected to an output layer 516, which outputs an output tensor 518. The output tensor 518 corresponds to the concentration ratio of each BTEX member of the measured family. In one application, the output layer 516 has four neurons 520-1 to 520-4, with each neuron corresponding to a single and unique member of the BTEX family. This means that the DNN 500 is trained to receive as input a spectrum that includes information about any number of members of the BTEX family (e.g., one member, two members, three members, or four members) and to distinguish between all these members and provide as output the presence of any of these members and their individual concentrations in the sample. In other words, the DNN 500 is capable to simultaneously process a spectrum that includes information from multiple members of the BTEX family, and to output the concentration of each member of the family at the same time.

FIG. 5 also shows a flow chart of the back propagation process 530 applied to the DNN 500. A calculated error (discussed later) is fed to an update process box 532 that updates the weights Wₖ and epoch k for each layer. After these parameters were updated in the DNN 500, a feed-forward calculation was performed in process box 534, which resulted in the estimation of the model results Modᵢ, where i corresponds to a single member of the BTEX family. Thus, if four members are considered to form the BTEX family, i takes the values 1, 2, 3, and 4. In process box 536, a difference between the Modᵢ results and the observation results Obsᵢ is calculated for a number N of datasets and the resulting error E is checked in process box 538 to determine whether the root mean squared error (RMSE) is smaller than a selected tolerance. If the answer is yes, the training process is terminated in process box 540. If the answer is no, the training returns to process box 530. Once the training is terminated, the last set of calculated weights Wₖ are used by the layers of the DNN 500 to estimate the presence of the various members of the BTEX family.

In one application, Python 3.8 software was utilized to build the prediction models. Linear, polynomial, sigmoid and radial-based function kernels were applied, with the latter being the most repeatable, with a tolerance of 0.001 for stopping criterion. For the DNN 500, hyper-parameter tuning was performed with RandomSearchCV (a known hyperparameter tuning tool) and 3 hidden layers of 64, 32 and 16 nodes, respectively. ReLU and Adamax (learning rate=0.001 and momentum=0.9) were selected for the activation function and optimizer, respectively. An optimizer is a function or algorithm that modifies the attributes of a neural network, such as weights and learning rate. Adamax is an extension to the Adam version of gradient descent that generalizes the approach to the infinite norm (max) and may result in a more effective optimization on some problems. The model was run on 2000 epochs with a batch size of 64, and its performance was monitored by mean-squared-error values. To avoid overfitting, the validation loss was monitored with a patience of 30 epochs.

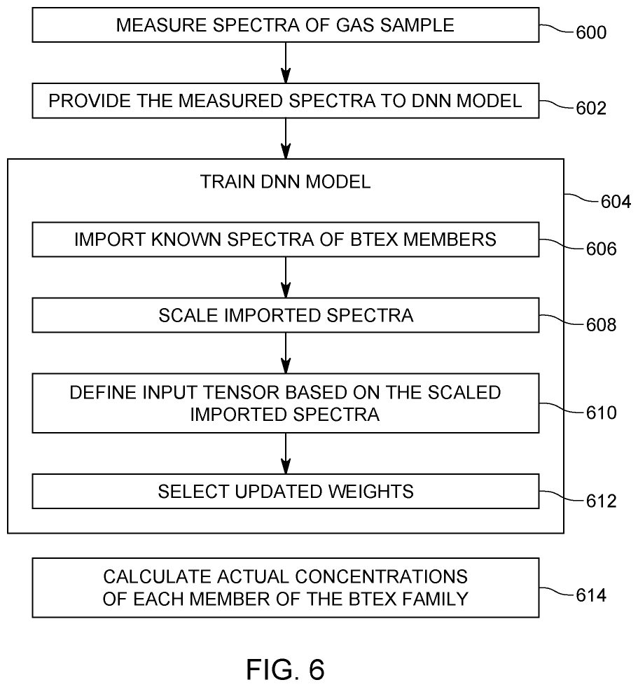

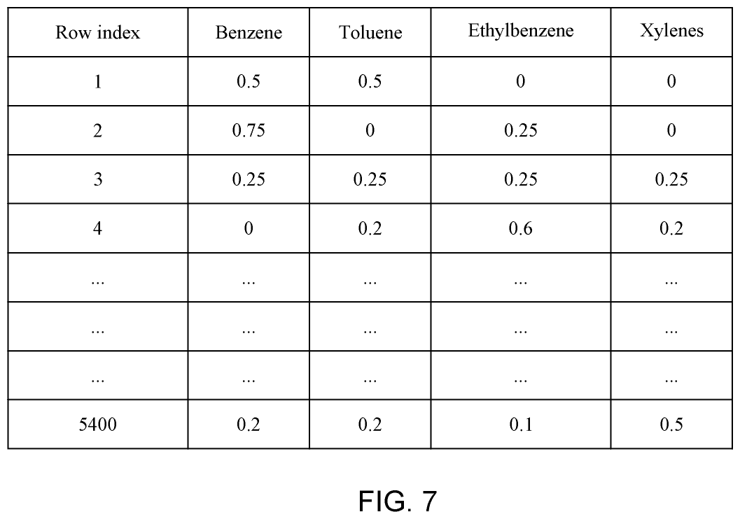

FIG. 6 is a flow chart of a method for determining the presence and the amounts of the members of the BTEX family, using the system 100. In step 600, system 100 performs one or more readings of the spectrum of an air sample that is present inside the measurement chamber 130, by using the laser beam 112 in the 3039.25-3040.5 cm⁻¹ range. The readings from the photosensor 140, which include information about the measured spectrum of the air sample, is provided in step 602 to the DNN 500. The DNN 500 is trained in step 604. This step may take place before receiving the measured spectrum in step 602. For this step, it is possible to use a total of 5400 inputs, which are randomly generated in a 5400×4 matrix as shown in FIG. 7, where the columns correspond to benzene, toluene, ethylbenzene and xylenes, respectively. The summation of the ratios in each row is constrained to unity.

The training step 604 may include a sub-step 606 of importing the absorbance spectra of benzene, toluene, ethylbenzene and xylenes from the PNNL database (or another database if desired). The total simulated absorbance in the 3039.25-3040.5 cm⁻¹ range is calculated by using the entries aᵢₖ in the table in FIG. 7 by using the following equation for row i=1,2,3, . . . , 5400:

$${A}_{i} = {\sum }_{k = 1}^{4} {a}_{i k} * {A}_{k} ,$$

where Aᵢ is the total absorbance of the i-th mixture, and Aₖ is the reference absorbance of the k-th BTEX species.

In addition, 132 measured absorbance spectra were added to this dataset. The measurements were performed on mixtures prepared in the lab by the inventors and the mixtures contained various concentrations of each BTEX member, within the range of 100-5000 ppm. Other measured absorbance spectra may be used.

In sub-step 608, the imported absorbance spectra are scaled by a min-max normalization so that the absorbance values are normalized and they are focused on the ratio of each species. The scaling is performed using the equation:

$${A}_{scaled} = \frac{A - {A}_{min}}{{A}_{max} - {A}_{min}} ,$$

where A_{scaled} is the resulting scaled absorbance, and Aₘᵢₙ and Aₘₐₓ are the minimum and maximum absorbance values among each simulated absorbance vector, respectively.

In sub-step 610, an input tensor is defined as the combination of the 5400 simulated and 132 measured, normalized/scaled BTEX spectra. The target tensor is the concentration ratio of each BTEX member. This data is fed into the DNN model 500 with 3 hidden layers and 64, 32 and 16 nodes, respectively. The data were randomly split into 80/20 train/test sets. The model performance was monitored by calculating RMSE. The flow chart in FIG. 5 shows the back propagation process followed in the DNN (BPNN) model.

In sub-step 612, the updated weights Wₖ for the plural layers of the DNN 500 are selected to be used for the data received in step 602. After the data received in step 602 is run through the DNN 500 with the selected weights from step 604, the output of the DNN model, which corresponds to the ratio of each of the BTEX member k, are used in step 614 to calculate the concentrations of each member of the BTEX family. In this embodiment, the concentration Xₖ for each member of the BTEX family is calculated based on equation:

$${χ}_{k} = {R}_{k} * \frac{{A}_{m e a s ured , total}}{{A}_{reference , k}}$$

where Rₖ is the ratio of each member to the entire BTEX family, A_{measured,total} is the total measured absorbance of the mixture obtained in step 600, and A_{reference,k} is the reference simulated absorbance of the k-th BTEX member with a specific concentration (taken from the table in FIG. 7).

Thus, the present method is capable to simultaneously measure the spectra of plural members of the BTEX family and then to provide the individual concentration of each member of the BTEX family. It is noted that the system discussed in [2] was not capable of simultaneously providing the individual concentration of each member of the BTEX family.

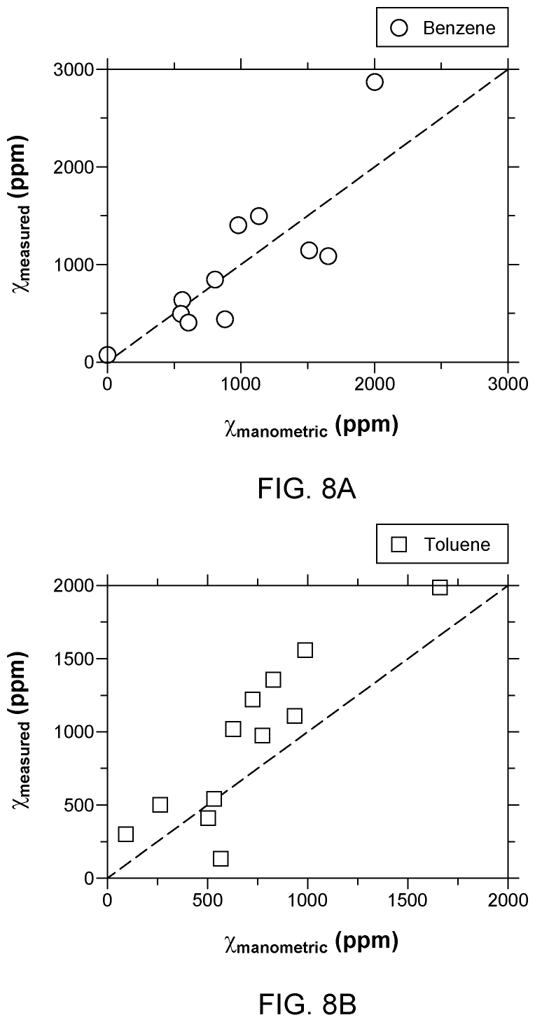

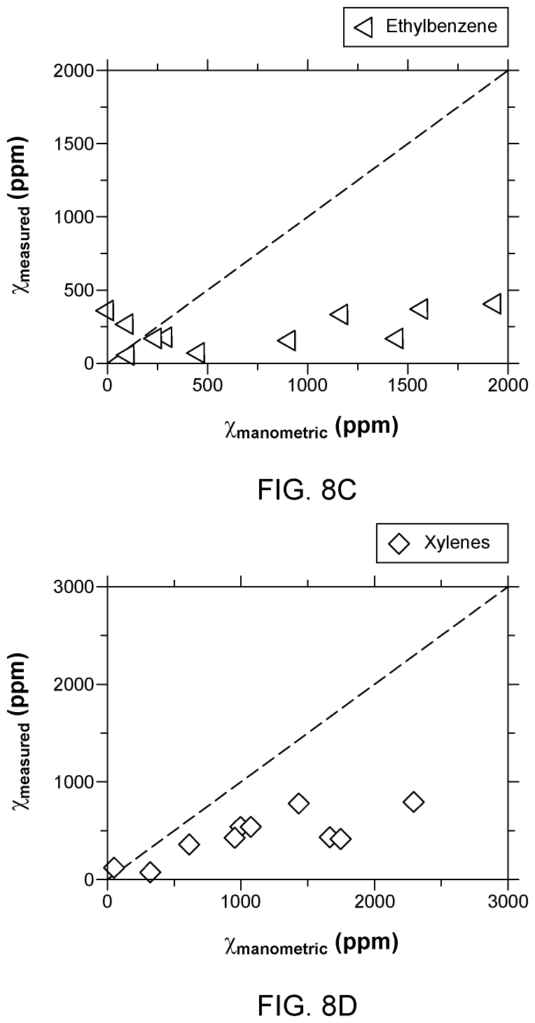

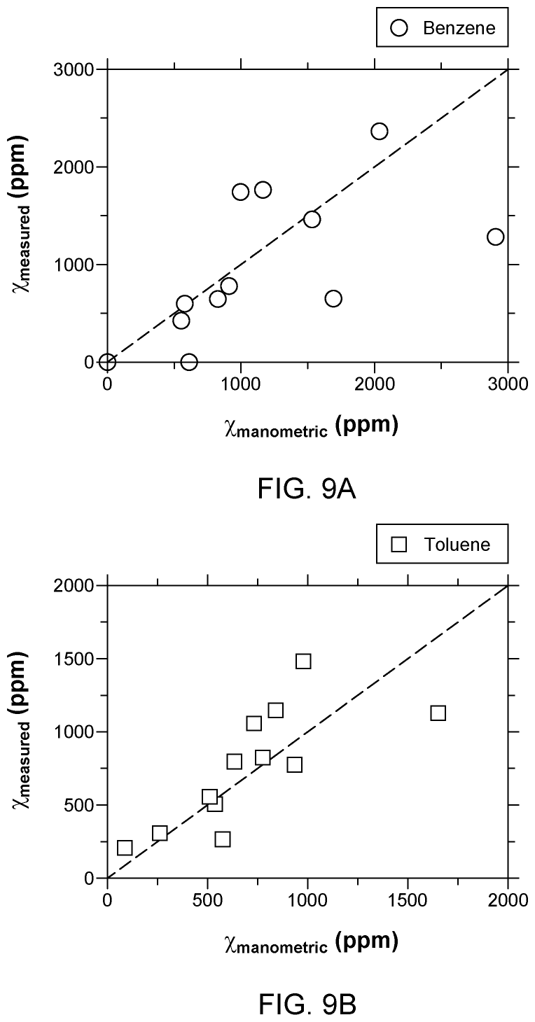

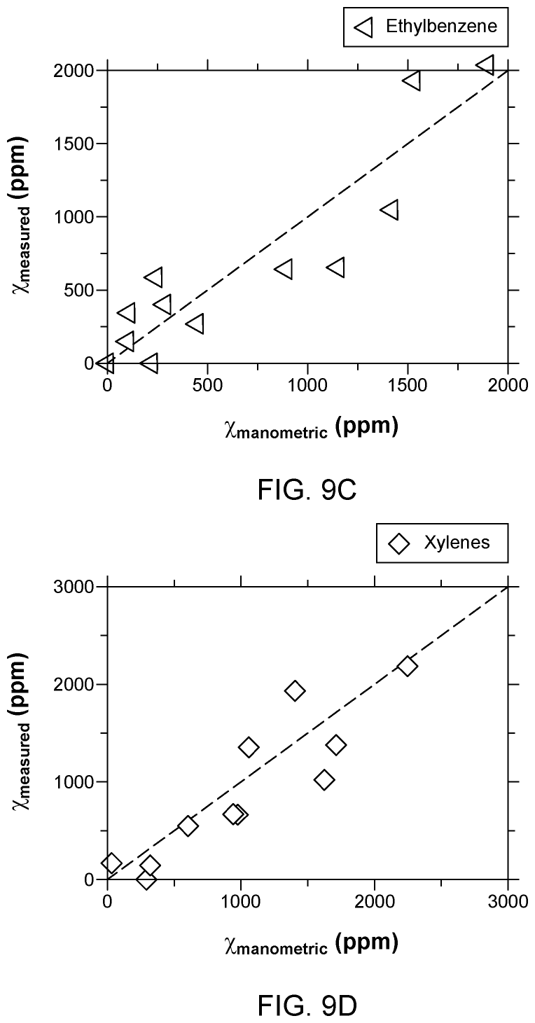

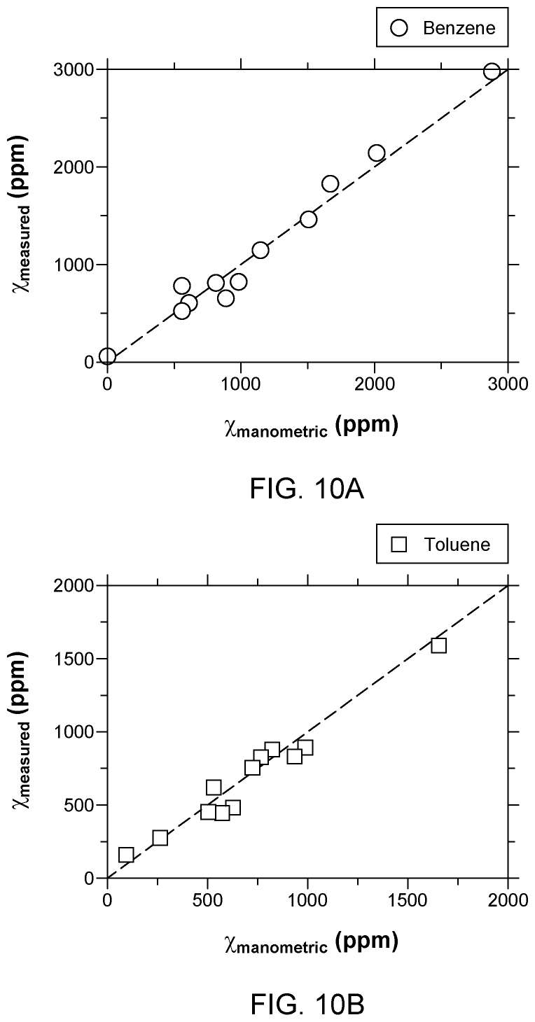

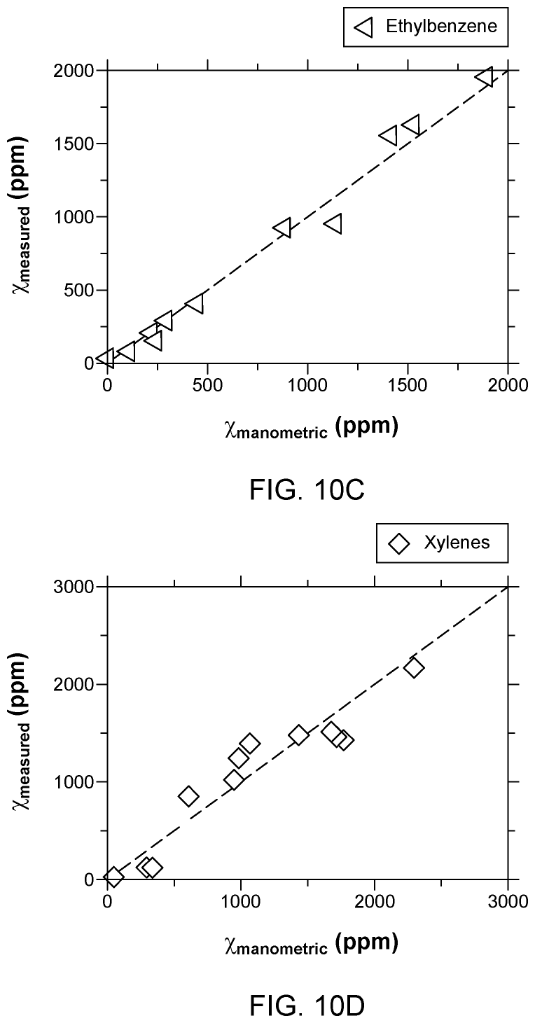

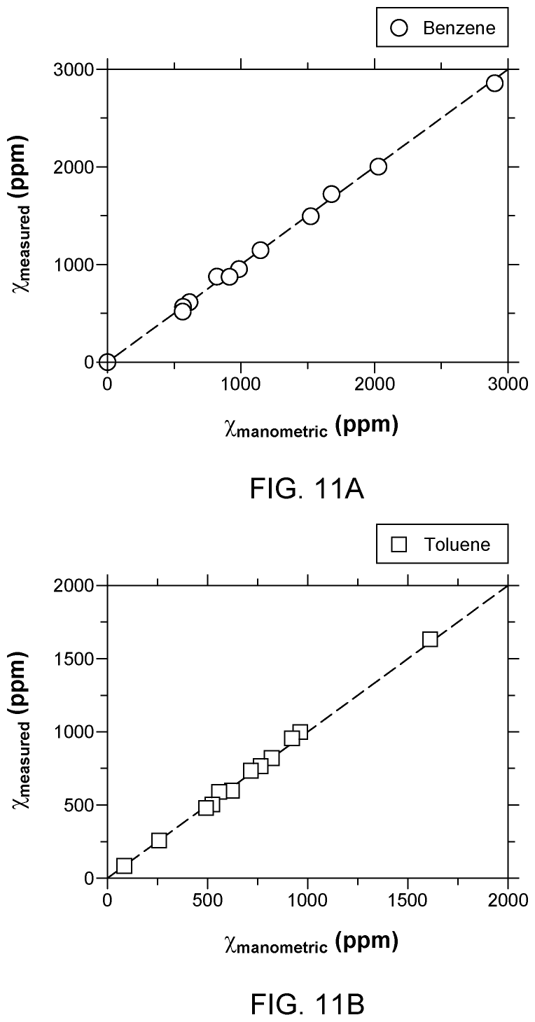

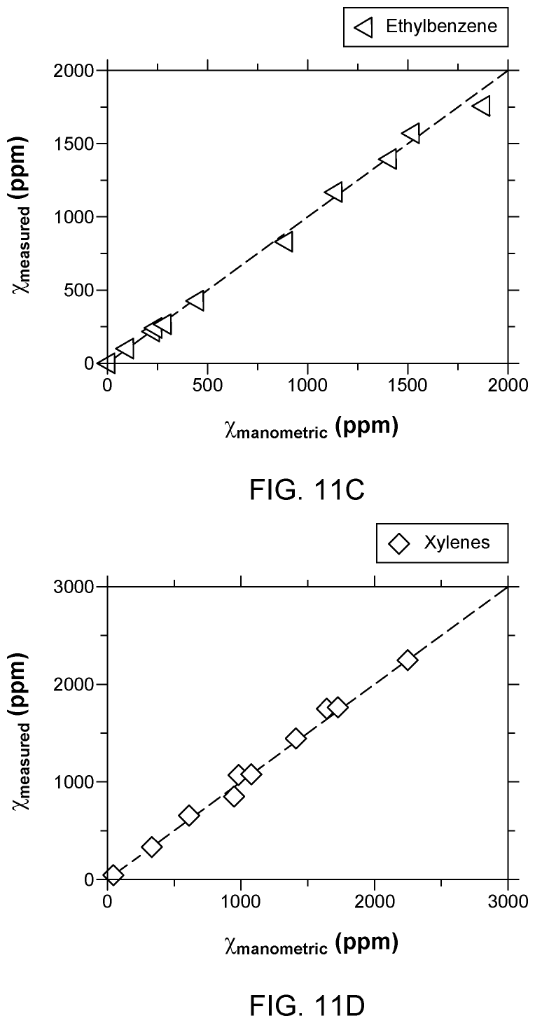

The portable, laser-based, selective BTEX sensing and analyzing system 100 was tested based on the method discussed in FIG. 6, to assess its performance, on 12 mixtures with varying concentrations of each of the BTEX members. The members' concentrations in the 12 mixtures were varied monometrically, and the measured total absorbance was analyzed with MLR, SVM, XGB, and DNN. FIGS. 8A to 8D show that the MLR method inadequately predicted the mole fractions of the BTEX members due to their highly similar reference spectra. The predictions of ethylbenzene and xylene slightly improved with the SVM approach as compared to MLR, as shown in FIGS. 9A to 9D. This is due to the higher flexibility of the SVM given by tweaking its hyper-parameters. Before training the model with XGB, careful selection of the number of estimators and learning rate is essential to prevent under/overfitting the model. The maximum depth of the tree, subsample ratio of columns and minimum weight required to create a new node in the tree were tuned. The XGB model significantly enhanced the prediction of BTEX mole fractions, as shown in FIGS. 10A to 10D, due to multiple hyper-parameter tuning, which gives the model more freedom when fitting the data as well as the fact that the XGB model utilizes first and second derivatives of the Taylor series of the optimization function. Finally, a random search algorithm was applied to tune the hyper-parameters of the DNN 500, including the number of hidden layers and nodes, activation functions, dropout layer, weight regularization, learning rate, momentum, number of epochs and batch size. An optimal DNN model 500 of three hidden layers 512-i with 64, 32, and 16 nodes, respectively, was utilized. The excellent agreement between the predicted and manometric BTEX molar fractions shown in FIGS. 11A to 11D validates the ability of the system 100 and DNN 500 to selectively and simultaneously measure BTEX molecules. This finding is in agreement with the cross-validation results shown in the table of FIG. 4. This analysis confirmed that the DNN 500 is the best model for this application due to the more expressive power of neural networks, and given by the high flexibility of tuning numerous hyper-parameters.

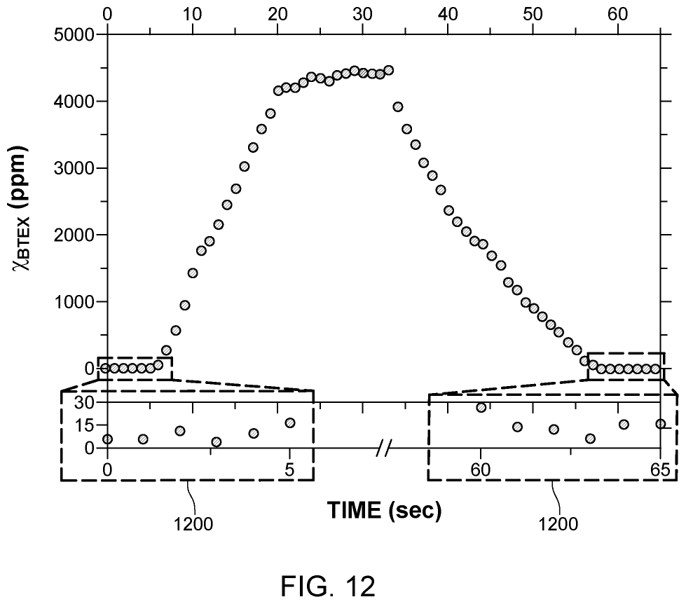

Real-time sensing is quite beneficial in practical applications where high temporal resolution is needed for trend evaluation. In this regard, FIG. 12 shows measurements performed on a 4400 ppm BTEX mixture (30% benzene, 30% toluene, 20% ethylbenzene and 20% xylenes) by flowing BTEX/N₂ continuously through the measurement chamber 130 of the system 100. Initially, the measurement chamber 130 was filled with air and the signal was recorded at a 1-second temporal resolution. The inlet 132 and outlet 134 of the measurement chamber were opened slightly but sufficiently so that the incoming gas sample replaced the air inside the chamber. The BTEX concentration increased up to 4400 ppm, and then decayed after flushing the chamber with air. To demonstrate the sensitivity of the system 100, the lower panels 1200 of FIG. 12 show zoomed-in data of the regions with only air inside the measurement chamber. The data shows that the detection limit of the sensor is ˜13±7 ppm of BTEX.

Thus, the portable system 100 is capable to perform selective and simultaneous BTEX measurements with an absorption-based laser sensor. The sensor was validated with gas samples by applying various machine learning algorithms. Best prediction results were obtained with the help of a DNN algorithm containing three hidden layers. Real-time monitoring of BTEX species was achieved with a temporal resolution of 1 second and minimum detection limit of 13 ppm.

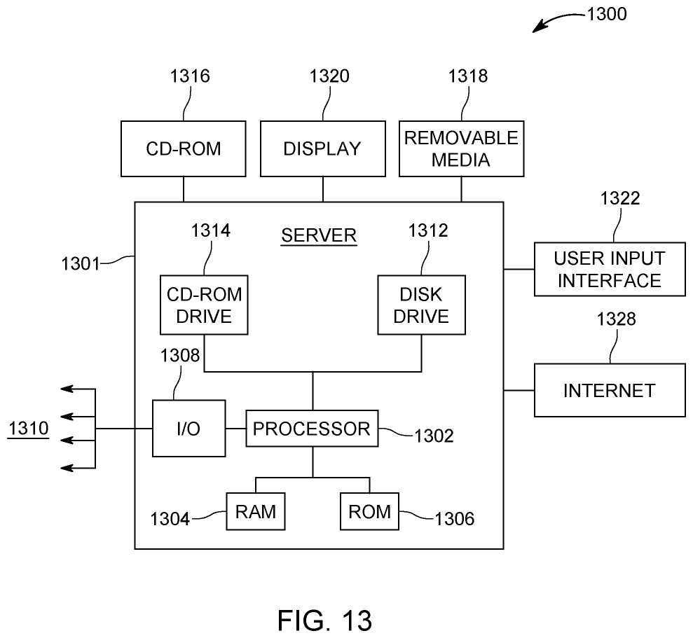

The above-discussed procedures and methods may be implemented in a computing device as illustrated in FIG. 13. Hardware, firmware, software or a combination thereof may be used to perform the various steps and operations described herein. Computing device 1300 suitable for performing the activities described in the exemplary embodiments may include a server 1301. Such a server 1301 may include a central processor (CPU) 1302 coupled to a random access memory (RAM) 1304 and to a read-only memory (ROM) 1306. ROM 1306 may also be other types of storage media to store programs, such as programmable ROM (PROM), erasable PROM (EPROM), etc. Processor 1302 may communicate with other internal and external components through input/output (I/O) circuitry 1308 and bussing 1310 to provide control signals and the like. Processor 1302 carries out a variety of functions as are known in the art, as dictated by software and/or firmware instructions.

Server 1301 may also include one or more data storage devices, including hard drives 1312, CD-ROM drives 1314 and other hardware capable of reading and/or storing information, such as DVD, etc. In one embodiment, software for carrying out the above-discussed steps may be stored and distributed on a CD-ROM or DVD 1316, a USB storage device 1318 or other form of media capable of portably storing information. These storage media may be inserted into, and read by, devices such as CD-ROM drive 1314, disk drive 1312, etc. Server 1301 may be coupled to a display 1320, which may be any type of known display or presentation screen, such as LCD, plasma display, cathode ray tube (CRT), etc. A user input interface 1322 is provided, including one or more user interface mechanisms such as a mouse, keyboard, microphone, touchpad, touch screen, voice-recognition system, etc.

Server 1301 may be coupled to other devices, such as sources, detectors, etc. The server may be part of a larger network configuration as in a global area network (GAN) such as the Internet 1328, which allows ultimate connection to various landline and/or mobile computing devices.

The disclosed embodiments provide a novel DNN-based, laser-based, detection and analysis system that is capable to simultaneously determine the presence and concentration of benzene, toluene, ethylbenzene, and xylenes. The embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Zhang, F., et al., Selective BTEX sensor based on a SnO2/V2O5 composite. Sensors and Actuators B: Chemical, 2013. 186: p. 126-131.
[2] U.S. Patent Application Publication No. 2022/0026354.
[3] Chen, T. and C. Guestrin. Xgboost: A scalable tree boosting system. in Proceedings of the 22nd acm sigkdd international conference on knowledge discovery and data mining. 2016.
[4] Montavon, G., W. Samek, and K.-R. Müller, Methods for interpreting and understanding deep neural networks. Digital Signal Processing, 2018. 73: p. 1-15.
[5] Raghu, M., et al. On the expressive power of deep neural networks. in international conference on machine learning. 2017. PMLR.

What is claimed is:

1. A laser-based detection and analysis system for detecting plural members of volatile organic compounds, the system comprising:
a measuring unit configured to simultaneously measure a spectrum of the plural members of the volatile organic compounds located in a measuring chamber, with a laser beam having a wavelength of about 3.3 μm; and
a data processing unit including a deep neural network, DNN, configured to process the spectrum measured by the measuring unit and to output an individual concentration of each of the plural members of the volatile organic compounds,
wherein the DNN is configured to update a weight Wₖ for each member of the plural members by using hidden layers having plural nodes, each node having an activation function and an optimizer.

2. The system of claim 1, wherein the hidden layers include only three layers, a first layer having 64 nodes, a second layer having 32 nodes, and a third layer having 16 nodes.

3. The system of claim 1, wherein the DNN has an output layer having only four nodes.

4. The system of claim 3, wherein a first node of the four nodes corresponds to benzene, a second node corresponds to toluene, a third node corresponds to ethylbenzene, and a fourth node corresponds to xylenes.

5. The system of claim 1, wherein the volatile organic compounds include benzene, toluene, ethylbenzene, and xylenes.

6. The system of claim 1, wherein the spectrum includes information about a mixture including benzene, toluene, ethylbenzene, and xylenes, and the data processing unit outputs the individual concentrations of each of the benzene, toluene, ethylbenzene, and xylenes.

7. The system of claim 1, wherein the DNN is trained with absorbance spectra of benzene, toluene, ethylbenzene, and xylenes in a 3039.25 to 3040.5 cm⁻¹ range for plural mixtures.

8. The system of claim 1, wherein the measuring chamber comprises:
a cavity having ZnSe windows at opposite ends, and one of the ZnSe windows is configured to receive the laser beam.

9. The system of claim 1, further comprising:
a laser device configured to generate the laser beam;
a photosensor configured to measure the spectrum of the plural members of the volatile organic compounds; and
a housing that houses the laser device, the measuring chamber, the photosensor, and the data processing unit,
wherein the housing is portable.

10. A laser-based detection and analysis system for simultaneously detecting benzene, toluene, ethylbenzene, and xylenes, the system comprising:
a laser device configured to emit a laser beam that includes a wavelength of 3.3 μm;
a measuring chamber configured to receive, at an internal cavity, ambient air and the laser beam, wherein the measuring chamber is configured to bounce the laser beam inside the internal cavity multiple times before exiting the measuring chamber;
a photosensor configured to receive an output laser beam from the measuring chamber; and
a data processing unit including a deep neural network, DNN, which is configured to receive a measurement from the photosensor and to simultaneously detect an amount of the benzene, toluene, ethylbenzene, and xylenes in the ambient air.

11. The system of claim 10, wherein the DNN is configured to update a weight Wₖ for each of the benzene, toluene, ethylbenzene, and xylenes by using hidden layers having plural nodes, each node having an activation function and an optimizer function.

12. The system of claim 10, wherein the DNN includes only three layers, a first layer having 64 nodes, a second layer having 32 nodes, and a third layer having 16 nodes.

13. The system of claim 10, wherein the DNN has an output layer having only four nodes.

14. The system of claim 13, wherein a first node of the four nodes corresponds to the benzene, a second node corresponds to the toluene, a third node corresponds to the ethylbenzene, and a fourth node corresponds to the xylenes.

15. The system of claim 10, wherein the DNN is trained with absorbance spectra of the benzene, toluene, ethylbenzene, and xylenes in a 3039.25 to 3040.5 cm⁻¹ range for plural mixtures.

16. The system of claim 10, wherein the measuring chamber comprises:
a cavity having ZnSe windows at opposite ends, and one of the ZnSe windows is configured to receive the laser beam.

17. The system of claim 10, further comprising:
a housing that houses the laser device, the measuring chamber, the photosensor, and the data processing unit,
wherein the housing is portable.

18. A method for simultaneously detecting plural members of volatile organic compounds, the method comprising:
simultaneously measuring, within a measuring unit, a spectrum of the plural members of the volatile organic compounds located in a measuring chamber, with a laser beam having a wavelength about 3.3 μm;
providing the measured spectrum to a data processing unit that includes a deep neural network, DNN; and
calculating at the DNN individual concentrations of each of the plural members of the volatile organic compounds,
wherein the DNN is configured to update a weight Wₖ for each member of the plural members by using hidden layers having plural nodes, each node having an activation function and an optimizer.

19. The method of claim 18, wherein the hidden layers include only three layers, a first layer having 64 nodes, a second layer having 32 nodes, and a third layer having 16 nodes.

20. The method of claim 18, wherein the DNN has an output layer having only four nodes, a first node of the four nodes outputs a concentration of benzene, a second node outputs a concentration of toluene, a third node outputs a concentration of ethylbenzene, and a fourth node outputs a concentration of xylenes.
